(19) United States
(12) Patent Application Publication
(10) Pub. No.: US 2004/0121016 A1
(43) Pub. Date: Jun. 24, 2004

(54) Functional composition, liquid composition and application method and apparatus therefor

(76) Inventors: Koichi Sato, Kanagawa (JP); Ikuo Nakazawa, Kanagawa (JP); Sakae Suda, Kanagawa (JP); Ryuji Higashi, Kanagawa (JP); Masayuki Ikegami, Kanagawa (JP); Keiichiro Tsubaki, Kanagawa (JP)

Correspondence Address: MORGAN & FINNEGAN, L.L.P., 345 PARK AVENUE, NEW YORK, NY, 10154, US

(21) Appl. No.: 10/732,697

(22) Filed: Dec. 9, 2003

(30) Foreign Application Priority Data
Jun. 14, 2002 (JP) 2002-174357
Jun. 16, 2003 (JP) 2003-171114

(51) Int. Cl. A61K 9/14

(52) U.S. Cl. 424/486

(57) ABSTRACT

A functional composition that contains a nonionic amphipathic block polymer, an ionic polymer, a functional substance such as a coloring material and a solvent. There are also provided a liquid composition that contains a nonionic amphipathic block polymer, an ionic polymer, a functional substance and a solvent, a liquid composition providing method to provide the above composition to a recording medium, a liquid composition providing apparatus which provides the above composition to a recording medium.

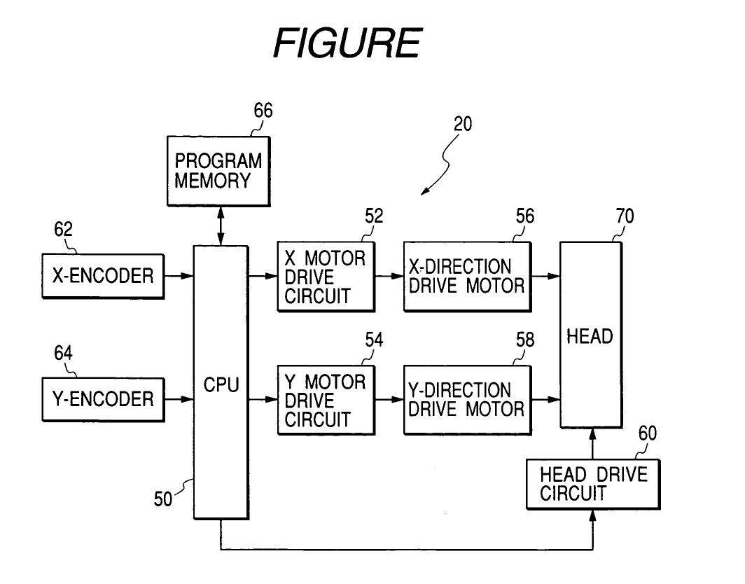

### BACKGROUND OF THE INVENTION

1. Field of the Invention

The present invention relates to functional compositions containing a polymer, a solvent and a functional substance performing a specific function. In particular, this invention relates to functional compositions being an aqueous dispersion, preferably ink compositions usable for printers, displays or the like, and to ink application methods and apparatuses using such ink compositions.

2. Related Background Art

As aqueous dispersion materials that contain granular functional materials, there are agricultural chemicals such as herbicides and insecticides, pharmaceuticals such as anticancer, antiallergic and antiinflammatory drugs. There are also aqueous dispersion materials containing colorants as granular functional materials, such as inks and toners. In recent years, digital print technology has been developing at a remarkable speed. This technology, represented by electrophotography technology and ink jet recording technology, is now getting indispensable more and more at office and home as an image forming technology.

Of the above technologies, the ink jet technology features compact size and low power consumption as a direct recording process. As the nozzles have become finer, higher image quality has been achieved. One example of such an ink jet recording method is a bubble jet method where the ink fed from an ink tank is heated with a heater provided in a nozzle, and boiling and bubbling of the ink eject the ink onto the recording medium for image formation. The other example is a method that ejects ink from a nozzle by using vibration of a piezo-electric device. Since these methods usually use aqueous dye solutions, blur may occur when different colors are superimposed, and a phenomenon called feathering may occur along the fibers of the recording medium such as paper. To improve such phenomena, use of pigment dispersion ink is studied in U.S. Pat. No. 5,085,698. However, further improvement is still desired.

### SUMMARY OF THE INVENTION

The object of this invention is to provide a functional composition that contains a polymer, a solvent and a functional substance performing a specific function. Preferably that composition has stimulation-responsive property. In particular, the object of this invention is to provide a functional composition being an aqueous pigment dispersion ink material where the solvent is water and the functional substance is a pigment, featuring high dispersion stability, less blur and feathering, excellent fixing properties, and good color properties.

The other object of this invention is to provide an ink composition usable for printers, displays or the like, and to provide an ink application method and apparatus using such an ink composition.

According to one aspect of the present invention, there is provided a functional composition comprising a nonionic amphipathic block polymer, an ionic polymer, a functional substance and a solvent.

According to another aspect of the present invention, there is provided a liquid composition comprising a nonionic amphipathic block polymer, an ionic polymer, a functional material and a solvent.

According to a still another aspect of the present invention, there is provided a method of providing a liquid composition to a recording medium comprising the steps of preparing the above liquid composition; and providing the composition to the recording medium.

According to a still another aspect of the present invention, there is provided an apparatus for providing a liquid composition to a recording medium comprising means for providing a liquid composition to a recording medium by adding energy to the liquid composition; and means for driving the composition providing means, wherein the liquid composition is the above-described composition.

### BRIEF DESCRIPTION OF THE DRAWING

FIGURE is a block diagram of an ink jet recording apparatus.

### DESCRIPTION OF THE INVENTION

The present invention is described in detail.

The first aspect of this invention is a functional composition that comprises a nonionic amphipathic block polymer, an ionic polymer, a functional substance performing a specific function, and a solvent.

One component characteristically used in this invention is a nonionic amphipathic block polymer. Because of its nonionicity, the nonionic amphipathic block polymer can disperse the functional substance, preferably a coloring material or a pigment in a solvent, especially in water, under both acidic and alkaline conditions. The nonionic amphipathic block polymer is required to have at least one hydrophobic block segment and at least one hydrophilic block segment. The block polymer may be any form of AB, ABA, ABC, ABCD and ABAC.

Concrete examples of the nonionic amphipathic block polymer include polystyrene-polyoxyethylene block polymer, polyoxyethylene-oxypropylene block polymer, polystyrene-polyacrylic acid block polymer, polystyrene-polyhydroxyethyl methacrylate block polymer, and poly(2-methoxyethyl vinyl ether)-poly(2-ethoxyethyl vinyl ether) block polymer.

The hydrophobic block segment of the nonionic amphipathic block polymer may comprise, for example, repeating (monomer) units having an alkyl group or an aromatic group. The hydrophilic block segment of the nonionic amphipathic block polymer may comprise, for example, monomer units having an alcohol substituent or a polyoxyethylene group.

From the viewpoint of satisfactory dispersion of a functional substance (in this invention preferably colorant or pigments) in a solvent, particularly in water, it is preferable that the nonionic amphipathic block polymer has a polyvinyl ether backbone structure. More preferably, the nonionic amphipathic block polymer is a polymer having a polyvinyl ether backbone structure whose monomer unit structure is represented by the following general formula (1):

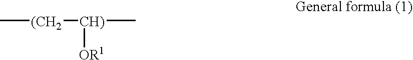

wherein R¹ is an alkyl group or one selected from the group consisting of —(CH(R²)—CH(R³)—O)_{I}—R⁴ and —(CH₂)ₘ—(O)ₙ—R⁴; and in the above groups, l is an integer selected from 1 to 18, m is an integer selected from 1 to 36, n is 0 or 1, R² and R³ are independently H or CH₃, R⁴ is selected from the group consisting of H, a straight-chain, branched-chain or cyclic alkyl group with 1 to 18 carbon atoms, an aromatic ring, —CO—CH═CH₂ and —CO—C(CH₃)═CH₂, and when R⁴ is not a hydrogen atom, any hydrogen atom on each carbon atom can be replaced with a straight-chain or branched-chain alkyl group of 1 to 4 carbon atoms, and a carbon atom in the aromatic ring can be replaced with a nitrogen atom.

Concrete examples of the structural formulae are as follows:

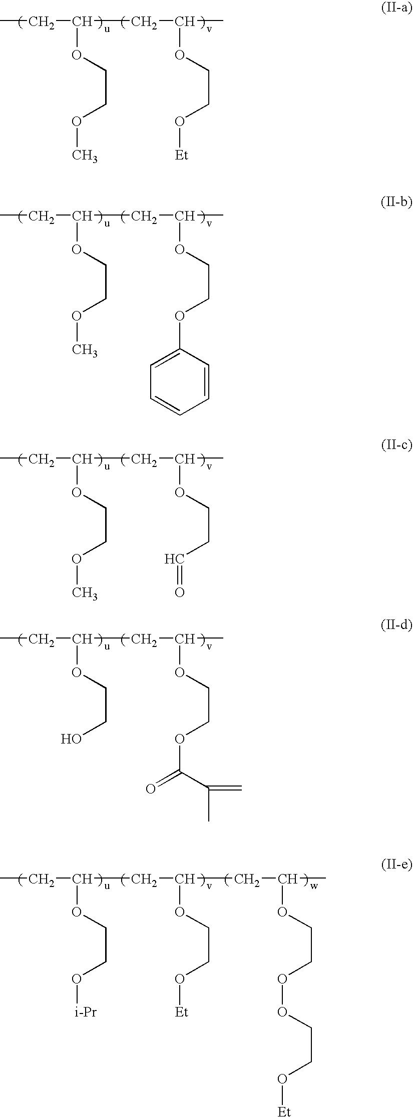

The block polymer having a polyvinyl ether backbone structure is not limited to those having 100% polyvinyl ether backbone, but includes those containing the polyvinyl ether backbone structure at 10 mole % or more. The content of polyvinyl ether backbone chain in the block polymer is preferably 10 mole % or more because of the flexibility of the polymer.

A number of processes have been proposed for production of polymers comprised of polyvinyl ether monomer units. Representative one is cation living polymerization proposed by Aoshima et al (Polymer Buretan, vol. 15, 417, 1986, Japanese Patent Application Laid-Open No. H11-322942). Cation living polymerization can produce various polymers with a precise length (molecular weight), and these polymers include homopolymers, copolymers comprised of plural monomer components, block polymers, graft polymers and graduation polymers. Various side chains can be introduced to the side chains of polyvinyl ether. Alternatively, cation polymerization process can be carried out using HI/I₂ or HCl/SnCl₄.

The number average molecular weight of the nonionic amphipathic block polymer used in this invention is preferably 100 or more and not more than 10,000,000, and more preferably 1,000 or more and not more than 1,000,000. The number average molecular weight of 100 or more is preferable because of preferable steric effect as a polymer, and the number average molecular weight of not more than 10,000,000 is preferable because of a proper viscosity.

Next, the functional substance performing a specific function used in the functional composition of this invention will be described. Typical examples of such a functional composition are compositions usable as agricultural chemicals such as herbicides and insecticides; compositions usable as pharmaceuticals such as anticancer, antiallergic and antiinflammatory agents; compositions usable as cosmetics such as lipstick, foundation, rouge and moisturizing cream; and compositions used as color materials such as ink and toner containing a colorant.

In this invention, the term “functional substance performing a specific function” (functional substance) means compounds or compositions that are contained in the functional composition of this invention and perform desired functions. For example, in the above-described agricultural chemicals, the functional substances are compounds having herbicidal or insecticidal activity. In the pharmaceuticals, they are compounds or pharmaceutical compositions that can alleviate or ameliorate the subject symptoms. In the cosmetics, they are essential substances of the desired products, for example, a moisturizing compound in a moisturizing cream. And in the ink and toners, they are dyes or granular materials such as pigments.

In this invention, preferably such a colorant is encapsulated in a block polymer. The colorant can be encapsulated in such a manner that, for example, the colorant dissolved or dispersed in a water-insoluble organic solvent is taken into micelles of the block polymer and then the organic solvent is distilled off. Alternatively, inclusion can be performed in such a manner that first the colorant and the block polymer are dissolved in an organic solvent followed by phase conversion with an aqueous solvent to form inclusion state, and then the remaining organic solvent is distilled off. The inclusion state of the colorant can be confirmed by instrumental analysis such as various types of electron microscopy, X-ray diffraction, etc. Alternatively, the inclusion state of the colorant can be confirmed by the colorant release from the micelles under conditions causing micelle destruction. As described above, it is preferable that the block polymer forms micelles, and therefore, it is preferable that the block polymer used in this invention is amphipathic.

The percentage of the colorant included in the block polymer with respect to the total amount of the colorant is preferably 90% or more, more preferably 95% or more, and much more preferably 98% or more. This ratio can be observed by instrumental analyses such as various types of electron microscopy, X-ray diffraction, etc. or by color density analysis of the colorant.

Specifically, pigments preferably used in this invention are pigments of black and three primary colors of cyan, magenta and yellow. Pigments of other colors, colorless or pale color pigments, and metalescent pigments may also be used. Pigments newly synthesized for this invention may also be used.

Commercially available black, cyan, magenta and yellow pigments are described below.

Examples of black pigments are, not limited to, Raven 1060 (Colombian Carbon), MOGUL-L (Cabot), Color Black FW1 (Degussa) and MA 100 (Mitsubishi Chemical).

Examples of cyan pigments are, not limited to, C. I. Pigment Blue-15:3, C. I. Pigment Blue-15:4 and C. I. Pigment Blue-16.

Examples of magenta pigments are, not limited to, C. I. Pigment Red-122, C. I. Pigment Red-123 and C. I. Pigment Red-146.

Examples of yellow pigments are, not limited to, C. I. Pigment Yellow-74, C. I. Pigment Yellow-128 and C. I. Pigment Yellow-129.

Further, pigments self-dispersible in water can also be used. There are two types of water-dispersible pigments: pigments adsorbing a polymer on the surface to utilize steric hindrance effect, and pigments utilizing electrostatic repulsion. Examples of commercially available water-dispersible pigments are CAB-0-JET 200 and CAB-0-JET 300 (Cabot) and Microjet Black CW-1 (Orient Chemical).

Concrete examples of dyes usable in this invention are water-soluble dyes such as direct dyes, acid dyes, basic dyes, reactive dyes, and food dyes, and water-insoluble colorants such as disperse dyes and oil soluble dyes.

Water-soluble dyes include: for example, direct dyes such as C. I. Direct Black-17, -62, -154, C. I. Direct Yellow-12, -87, -142, C. I. Direct Red-1, -62, -243, C. I. Direct Blue-6, -78, -199, C. I. Direct Orange-34, -60, C. I. Direct Violet-47, -48, C. I. Direct Brown-109 and C. I. Direct Green-59; acid dyes such as C. I. Acid Black-2, -52, -208, C. I. Acid Yellow-11, -29, -71, C. I. Acid Red-1, -52, -317, C. I. Acid Blue-9, -93, -254, C. I. Acid Orange-7, -19 and C. I. Acid Violet-49; reactive dyes such as C. I. Reactive Black-1, -23, -39, C. I. Reactive Yellow-2, -77, -163, C. I. Reactive Red-3, -111, -221, C. I. Reactive Blue-2, -101, -217, C. I. Reactive Orange-5,-74, -99, C. I. Reactive Violet-1, -24, -38, C. I. Reactive Green-5, -15, -23 and C. I. Reactive Brown-2, -18, -33; C. I. Basic Black-2, C. I. Basic Red-1, -12, -27, C. I. Basic Blue-1, -24, C. I. Basic Violet-7, -14, -27; and C. I. Food Black-1, -2.

Examples of commercially available oil-soluble dyes for the various color types are listed below.

Examples of black oil-soluble dyes are, not limited to, C. I. Solvent Black-3, -22:1, and -50.

Examples of yellow oil-soluble dyes are, not limited to, C. I. Solvent Yellow-1, -25:1, and -172.

Examples of orange oil-soluble dyes are, not limited to, C. I. Solvent Orange-1, -40:1, and -99.

Examples of red oil-soluble dyes are, not limited to, C. I. Solvent Red-1, -111, and -229.

Examples of violet oil-soluble dyes are, not limited to, C. I. Solvent Violet-2, -11, and -47.

Examples of blue oil-soluble dyes are, not limited to, C. I. Solvent Blue-2, -43, and -134.

Examples of green oil-soluble dyes are, not limited to, C. I. Solvent Green-1, -20, and -33.

Examples of brown oil-soluble dyes are, not limited to, C. I. Solvent Brown-1, -12, and -58.

Next, the solvents used in this invention are described.

The solvents used in this invention include: for example, organic solvents such as various types of straight, branched or cyclic aliphatic hydrocarbons, aromatic hydrocarbons, hetero aromatic hydrocarbons and halogen-containing solvents; aqueous solvents; and water. Particularly in the composition of this invention, water and aqueous solvents can be used singly or in combination.

Examples of aqueous solvents are polyhydric alcohols such as ethylene glycol, diethylene glycol, triethylene glycol, polyethylene glycol, propylene glycol, polypropylene glycol and glycerol; polyhydric alcohol ethers such as ethylene glycol monomethyl ether, ethylene glycol monoethyl ether, ethylene glycol monobutyl ether, diethylene glycol monoethyl ether and diethylene glycol monobutyl ether; and nitrogen solvents such as N-methyl-2-pyrrolidone, substituted pyrrolidone and triethanolamine. When the composition is used as an ink, monohydric alcohols such as methanol, ethanol and isopropyl alcohol may be used to speed up drying on paper.

Next, the ionic polymer used in this invention is described.

The ionic polymer is a polymer having an ionic functional group such as carboxylic acid salt, sulfonic acid salt, phosphoric acid salt or hydrochloride salt of amine. The ionic polymer preferably has at least 1 mol % or more of such ionic functional group, more preferably 10 mol % or more. If the ionic polymer has less than 1 mol % of ionic functional groups, the properties resulting from the ionic functional groups may be insufficient.

Concrete examples of ionic polymers are metal salts of poly(meth)acrylic acid; metal salts of polyvinyl sulfonic acid, polyvinyl benzenesulfonic acid, poly(meth)acrylamide alkyl sulfonic acid and polymaleic acid; or copolymers obtained using, as main components, the monomeric components constituting the above polymer compounds; metal salts of the polyvinyl alcohol—polyacrylic acid complex; metal salts of carboxymethyl cellulose; metal salts of carboxyethyl cellulose; or copolymers or polymer blends obtained using, as main components, the above polymer compounds. The above ionic polymer is preferably an amphipathic block polymer. Examples of ionic amphipathic block polymers are polystyrene-polysodium acrylate block polymer, polystyrene-polyvinyl pyridine hydrochloride block polymer, polymethyl methacrylate-polyvinyl pyridine hydrochloride block polymer and polystyrene-polysodium methacrylate block polymer.

The number average molecular weight of the ionic polymer used in this invention is preferably 100 or more and 10,000,000 or less, more preferably 1,000 or more and 1,000,000 or less. The ionic polymer with a number average molecular weight of 100 or more is preferable because it has a preferable steric effect as a polymer, whereas the ionic polymer with a number average molecular weight of 10,000,000 or less is also preferable because it has a proper viscosity.

One preferable example of the ionic polymer used in this invention is a block polymer, more preferably an amphipathic block polymer. One preferable example of such ionic amphipathic block polymer is an ionic block polymer having the same repeated structural unit as at least one block segment of the nonionic block polymer, which is used in this invention in combination with the ionic block polymer because uniform micelles can be formed by the nonionic block polymer and the ionic block polymer due to the closeness in nature of the two polymers. An ionic block polymer having the same monomer units as two block segments of the nonionic block polymer is a more preferable example. Accordingly, the ionic block polymer preferably used in this invention preferably has the above described polyvinyl ether repeated structural unit, and preferable examples of the ionic block polymer are ionic block polymers that have monomer units having the above described general formula (1), or the above described concrete examples of monomer units. And examples of ionic monomer units are represented by the following general formula (2) or (3).

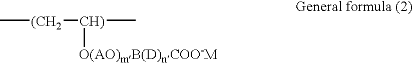

wherein A represents a straight-chain or branched-chain alkylene or substituted alkylene group with 1 to 15 carbon atoms, m′ is an integer of 0 to 30, and when m′ is 2 or more, each A may represent different alkylene groups; B represents a single bond or substituted alkylene group, D is an aromatic ring structure, n′ is an integer of 0 to 10, and when n′ is 2 or more, each D may represent different aromatic ring structures; M represents a monovalent or polyvalent metal cation.

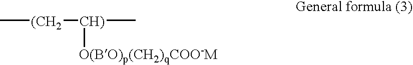

wherein B′ represents a straight-chain or branched-chain alkylene or substituted alkylene group with 1 to 15 carbon atoms, p is an integer of 0 to 30, and when p is 2 or more, each B′ may represent different alkylene groups; q represents an integer of 2 to 30; M represents a monovalent or polyvalent metal cation.

Concrete examples of monomer units having the general formula (2) or (3) are as follows.

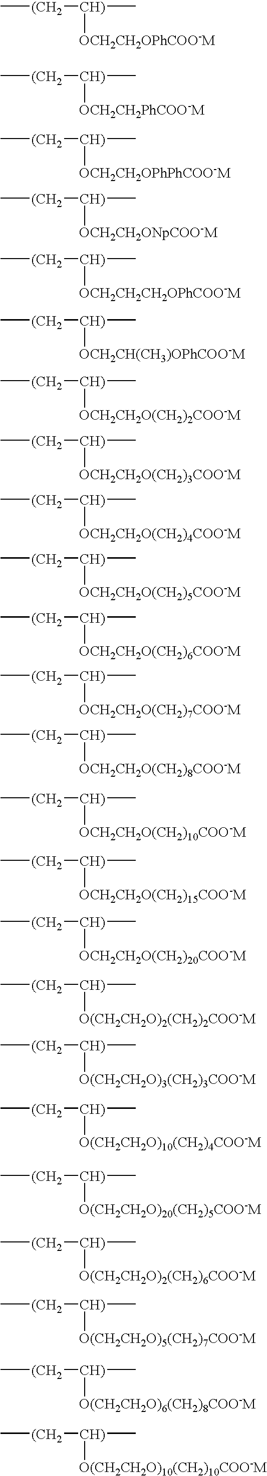

Next, the contents of the nonionic amphipathic block polymer, ionic polymer, functional substance performing specific function and solvent in the functional composition of this invention are described.

The content of the nonionic amphipathic block polymer used in the functional composition is 0.1 wt % or more but not more than 90 wt %, preferably 1 wt % or more and not more than 70 wt %, and more preferably 1 wt % or more and not more than 30 wt %. The content is 0.1 wt % or more for full exertion of effect of the block polymer. The content is 90 wt % or less, not to inhibit the properties of other components.

The content of the ionic polymer used in the functional composition is 0.1 wt % or more but not more than 90 wt %, preferably 1 wt % or more and not more than 70 wt %, and more preferably 1 wt % or more and not more than 30 wt %. The content is 0.1 wt % or more, because the effect of the polymer is fully exerted in such a range. The content is 90 wt % or less, because the polymer would not inhibit the properties of other components.

The content of the functional substance used in the functional composition is 0.1 wt % or more but not more than 90 wt %, preferably 1 wt % or more and not more than 50 wt %, and more preferably 2 wt % or more and not more than 30 wt %. The content is 0.1 wt % or more, because the effect of the substance is fully exerted. The content is 90 wt % or less, because it would not inhibit the properties of other components.

The content of the solvent used in the functional composition is 5 wt % or more but not more than 99 wt %, preferably 10 wt % or more and not more than 90 wt %, and more preferably 30 wt % or more and not more than 90 wt %. The content is 5 wt % or more, because the effect of the solvent is fully exerted. The content is 99 wt % or less, because the solvent would not inhibit the properties of other components.

The composition of this invention may contain appropriate additives other than the above-described compounds.

One characteristic of this invention is the coexistence of a nonionic amphipathic block polymer and an ionic polymer, which allows more stable dispersion of the functional substance, preferably a coloring material, and enables smaller particle size of the functional substance, in comparison with the case where only one of them is present.

Next, the second aspect of this invention, a stimulation-responsive composition, is described.

The stimulation-responsive composition of this invention is a composition made up as described above and further has a stimulation-responsiveness. The composition of this invention can change its state (properties) in response to various types of stimulation. Changes in state include: for example, phase change from sol to gel, or from solution to solid; and changes in chemical structure. In this invention, types of stimulation include: temperature change; application of electric field; exposure to light rays or electromagnetic wave such as ultraviolet ray, visible ray and infrared ray; pH change of the composition; addition of chemical substances; and concentration change of the composition.

The term “stimulation-responsiveness” used herein means that the composition of this invention changes its properties in response to the stimulation applied thereto. Specifically, the stimulation-responsiveness means that the composition of this invention markedly changes its form or physical properties in response to the applied stimulation or environmental change such as electromagnetic waves, electric field, temperature change, pH change, addition of a chemical substance or concentration change of the composition. The properties of the composition to be changed can be selected depending on the purpose for which the composition is used. For example, when used in pharmaceuticals, the chemical structure, in particular, the chemical bonding in the compound changes in response to the applied stimulation to release the functional substance. When used in cosmetics, phase change in the composition in response to the applied stimulation will work to prevent evaporation of a functional substance. Further, when used as a coloring material, phase change of the composition (e.g. from sol to gel) in response to the applied stimulation will work to improve the fixation to the recording medium.

Examples of stimulation-responsiveness preferable in this invention are as follows.

First, there is stimulation-responsiveness to temperature change in the composition encompassing the phase transition temperature of the composition. Secondly, there is stimulation-responsiveness to exposure to electromagnetic wave of preferably 100 to 800 nm. Thirdly, there is stimulation-responsiveness to pH change in the composition in the range of from 3 to 12. Fourthly, there is stimulation-responsiveness to concentration change in the composition. Such stimulation is caused, for example, when the solvent in the composition is evaporated or absorbed or when the concentration of polymers dissolved in the composition is changed. In this case, the concentration change preferably occurs to span the phase transition concentration of the composition. Another example is stimulation-responsiveness to addition of a certain type of chemical substance to the composition, with which the composition causes reaction or physical change causing thickening or aggregattion. Two types or more of stimulation described above may be applied in combination to the composition of this invention.

As described above, the composition of this invention finds its way into many applications such as agricultural chemicals, pharmaceuticals, cosmetics and colorant; however, the composition is particularly preferably used as an ink material containing a pigment or a dye and water as a solvent. The use of the composition of this invention allows improvement in various properties of ink including fixing properties.

Preferably the polymer materials for the composition of this invention are designed to have such stimulation-responsiveness. In this respect, the nonionic block polymer having a polyvinyl ether backbone chain described above is preferably used. Such block polymers are preferably used because they are responsive to temperature stimulation and chemical substances such as carboxylic acid in a solvent, particularly in water, and can satisfactorily disperse the functional substance such as pigments. In addition, the ionic polymer of the invention has a tendency to exhibit stimulation-responsiveness to an appropriate counter ion and preferably designed as such. Preferably, the ionic polymer and the nonionic block polymer have different stimulation-responsiveness.

Next described is the third aspect of this invention, an ink composition usable as an ink jet ink.

The ink jet ink of this invention is produced by: for example, adding a pigment, a nonionic amphipathic block polymer having a function as a dispersant and an ionic polymer to water and a water-soluble solvent; dispersing the mixture with a disperser; removing coarse particles by centrifugation etc.; adding water or a solvent and additives; followed by stirring, mixing and filtration.

Examples of dispersing machines used in this invention are an ultrasonic homogenizer, a laboratory homogenizer, a colloid mill, a jet mill and a ball mill, and these may be used individually or in combination. Even in cases where a self-dispersing pigment is used, the ink jet ink of this invention can be produced by the same process as above. One preferred embodiment of this invention is an ink composition used as ink jet ink that has stimulation-responsiveness.

The fourth aspect of this invention is an image forming method in which recording is carried out by ejecting ink from an ink ejecting port and providing it to a recording medium, characterized by using the above composition as ink. The aqueous dispersion ink of this invention can be used in various image forming apparatuses of various printing methods, ink jet method and electrophotographic method to form images. The aqueous dispersion ink of this invention can be used as an ink jet ink, for examples, in the following embodiments. Examples of the usage of the ink jet ink of this invention as stimulation-responsive ink are given below. The ink can aggregate or thicken by the stimulation of (a) to (d).

(a) Ink Responsive to Temperature Stimulation

The ink jet ink of this invention ejected from an ink tank and attached on a recording medium undergoes temperature stimulation due to the difference in temperature between the ink tank and the recording medium, and the temperature stimulation causes phase change in the ink jet ink of this invention, resulting in rapid thickening of the ink jet ink and aggregation of insoluble components of the ink.

(b) Ink Responsive to Electromagnetic Wave Stimulation

The ink jet ink of this invention, ejected from an ink tank being a dark room and attached on a recording medium, undergoes electromagnetic wave stimulation when it is exposed to visible rays after ejection or irradiated with electromagnetic wave emitted from the electromagnetic wave emission part provided in the ink jet recording apparatus, which causes polymerization of polymerizable functional groups contained in the ink jet ink of this invention, resulting in thickening of the ink jet ink or aggregation of the insoluble components of the ink.

(c) Ink Responsive to pH Change Stimulation

The ink jet ink of this invention ejected from an ink tank and attached on a recording medium undergoes pH change stimulation when its pH changes under the effect of the recording medium to cause phase change in the ink jet ink of this invention, resulting in thickening of the ink jet ink or aggregation of the insoluble components of the ink.

(d) Ink Responsive to Concentration Change Stimulation

The concentration of the ink in an ink tank will change when the ink is ejected and attached to the recording medium because the water and aqueous solvent contained in the ink evaporate and are absorbed by the recording medium, which causes phase change in the ink jet ink of this invention, resulting in the rapid thickening of the ink jet ink or aggregation of the insoluble components of the ink.

These modifications of ink properties allows the improvement of blur and feathering in print as well as the excellent fixing properties of ink. The change in ink properties are not limited to the above-described thickening of ink and aggregation of the insoluble components of the ink.

To give stimulation to the ink jet ink of this invention, various methods can be applied. One preferred method is to mix or contact the above-described stimulation-responsive ink with a stimulant composition. For example, the ink jet method can be applied to mix the pH-responsive ink described in (c) with a composition of corresponding pH. As described in Japanese Patent Application Laid-Open No. S64-63185, a stimulant composition is applied to the image-forming area of the recording medium using an ink jet head. Alternatively, as described in Japanese Patent Application Laid-Open No. H8-216392, superior images can be formed by controlling the amount of the stimulant composition.

A stimulant composition can be an ink that contains a dye or a pigment. For example, if any one of cyan-, magenta-, yellow- and black-ink for color ink jet system is used as a stimulant and any other one of CMYK inks as a stimulation-responsive ink, blur in print can be improved. No restriction is imposed on which one of CMYK ink should be used as stimulation-responsive ink and which other one of CMYK ink should be used as a stimulus, and various combinations are possible. In this invention, any one of the possible combinations can be used and the selection of a combination is not limited. Realizing various combinations also means that variation of product design is widened and demands of users can be met. It goes without saying that it is a key point to provide various types of stimulation-responsiveness to the respective inks in order to enable various combinations. In this respect, the use of the composition of this invention described above is beneficial.

The stimulation-responsive ink composition of this invention contains at least a nonionic amphipathic block polymer and an ionic polymer; therefore, the composition can have plural types of stimulation-responsiveness. This increases the number of the ink combinations that can improve color blur in print to realize images of still higher quality. In this case, too, types of the stimulant composition and stimulation-responsive ink can be selected from all the above described stimulation-response patterns and are not limited to specific ones. It is possible, of course, to perform recording with a stimulation providing solution that contains no coloring material in proper timing before or after recording with ink.

It is also preferable to provide a recording medium with a stimulation-giving mechanism. For example, recording may be performed on acid paper with acid responsive ink, a kind of pH responsive ink. In this case, the recording medium stimulates the stimulation responsive ink of this invention. Such a recording medium is also included in this invention. In other words, this invention also relates to a recording medium having a function of stimulating the stimulation responsive ink of this invention. In this invention, the recording medium may take any known forms. For example, the recording medium may be plain paper, thermal paper or acid paper.

Next described is the fifth aspect of this invention, an image forming apparatus to perform recording by ejecting ink from its ink ejecting portion and providing the same on a recording medium, characterized by using the above described ink.

The ink jet printer using the ink jet ink of this invention may adopt various ink jet recording methods, such as the piezo ink jet method which employs a piezo-electric device and the thermal ink jet method in which recording is performed utilizing bubbles generated by heat energy. In the following the ink jet recording apparatus will be described with reference to FIGURE. However, it is to be understood that FIGURE is shown by way of example and is not intended to limit this invention.

FIGURE is a block diagram illustrating the structure of an ink-jet recording apparatus.

The apparatus shown in FIGURE does recording on a recording medium moving the head. Referring to FIGURE, the motors 56 and 58 responsible for driving the head 70 in the X and Y directions, respectively, are linked to the CPU 50, which commands all actions in the apparatus, via the circuits 52 and 54 for driving each motor. The CPU 50 instructs the motors 56 and 58 via the circuits 52 and 54 to drive the head 70 in the X and Y directions to a given position on the recording medium.

As shown in FIGURE, the head-driving circuit 60 is also linked to the head 70, in addition to the motors 56 and 58 for driving the head in the respective X and Y direction, to drive the head 70 for a given action, e.g., discharging the ink, following the instruction from the CPU 50. The CPU 50 receives information of the head 70 position from the X encoder 62 and Y encoder 64 responsible for detecting the head position, which are also linked to the CPU 50. A control program is inputted in the program memory 66. The CPU 50 drives the head 70, based on the control program and position information from the X encoder 62 and Y encoder 64, to a desired position on the recording medium, and instructs the head to discharge the ink at that position. The apparatus forms a desired image on the recording medium in the above manner. For the image-forming apparatus which can hold 2 or more types of inks for ink-jet, the above procedure is repeated necessary times with each ink to produce a given image on the recording medium.

The head 70 can be also moved after it has discharged the ink as required, to a position where a means (not shown) for removing surplus ink deposited on the head is provided, to be cleaned by proper wiping means. Specific cleaning means may be selected from the conventional means used for the above purpose.

On completion of the above image-forming procedure, the recorded medium is replaced by a new medium by a recording medium conveying mechanism, which is not shown.

The above embodiment can be modified or varied within scope of the present invention. For example, the head 70 is moved in the X and Y directions in the above embodiment. However, it may be designed to move only in X (or Y) direction with the recording medium moving in Y (or X) direction, to form image.

According to the present invention, an ink jet recording head provided with means (e.g., an electro-thermal converting element or a laser) for generating thermal energy to discharge the ink brings the excellent effect. Such a system can produce precision images. The image quality can be further improved when the ink composition of the present invention is used in thermal ink jet recording.

The representative structures of and working principles for the apparatus provided with a means for generating thermal energy, e.g., the one described above, are preferably based on the basic principles disclosed by, e.g., U.S. Pat. Nos. 4,723,129 and 4,740,796. These apparatuses are applicable either to the so-called on-demand or continuous type. The apparatus of the present invention is particularly effective when applied to the on-demand type, because the liquid is securely held, and at least one type of driving signal, which corresponds to the discharge information, is applied to the electro-thermal converting element positioned in the flow path to generate thermal energy and increase temperature rapidly enough to cause at least nuclear boiling. This thermal energy causes film boiling on the heater board in the head, on which bubbles are formed by the action of heat according to the signals in one-to-one response. The liquid is discharged from the discharge port by expansion/shrinkage of the foams, to form at least one droplet. The pulsed driving signal is more preferable, because it immediately causes expansion/shrinkage of the foams, achieving quicker response for discharging the liquid. The pulse driving signals described in U.S. Pat. No. 4,463,359 or 4,345,262 are preferable. More excellent discharge can be done under the conditions concerning temperature increasing rate on the heater described in U.S. Pat. No. 4,313,124.

In addition to the head structure described in above Patent documents comprising a discharge orifice, an electro-thermal converting element, and a flow path that is straight or right angle, another structure disclosed in U.S. Pat. No. 4,558,333 or 4,459,600 is also included in the present invention where the heater is provided in a curved region. All of these structures are within scope of the present invention. Moreover, the present invention is also effective with a structure with two or more electro-thermal converters and a common slit working as the discharge port for these converters, or a structure provided with an opening for absorbing pressure waves caused by the thermal energy as the discharge port. In short, the present invention can discharge the ink securely and efficiently by the head of any structure.

The image-forming apparatus of the present invention can also efficiently work, when provided with a full-line type head, which covers the maximum width of the recording medium. The head structure is not limited. For example, the maximum width can be covered by a combination of two or more heads, or by a single head.

Moreover, the apparatus of the present invention can also efficiently work, when provided with a serial type head, a head fixed on the apparatus body, or a chip type head that is exchangeably mounted on the apparatus body and electrically linked to and supplied with the ink from the apparatus body. The apparatus of the present invention may be further provided with a means for removing liquid droplets. Such an apparatus can realize still more favorable discharging effect.

The apparatus of the present invention may have a structure provided with an auxiliary means. Such a structure is preferable, because it can further stabilize the effect of the present invention. The specific examples of these auxiliary means include a capping means for the head, pressurizing or evacuating means, preheating means for another electro-thermal converting element, a different heating element, or a combination thereof, and a preliminarily discharging means other than the ink discharging means.

The apparatus most effective for the present invention is that utilizing film boiling as described above.

Each port for the head of the present invention preferably discharges 0.1 to 100 picoliters of the ink.

The composition of this invention can be used in indirect recording apparatuses which employ a recording method in which print is first done on an intermediate transferring medium with ink and then the print was transferred to a recording medium such as paper. The composition of this invention can also be used in recording apparatuses utilizing an intermediate transferring medium in accordance with direct recording method.

### EXAMPLES

In the following this invention will be described in detail in terms of several examples; however, it is to be understood that these examples are not intended to limit this invention.

### Example 1

### Synthesis of Nonionic Amphipathic Block Polymer

### Preparation of Monomers

2-methoxyethyl vinyl ether (hereinafter abbreviated as MOVE) and 2-ethoxyethyl vinyl ether (hereinafter abbreviated as EOVE) were synthesized separately by refluxing a mixture of 2-chloroethyl vinyl ether and sodium methoxide or a mixture of 2-chloroethyl vinyl ether and sodium ethoxide, respectively, in the presence of tetrabutylammonium iodide catalyst (refer to U.S. Pat. No. 3,062,892 Specification).

### Synthesis of AB Diblock Polymer Consisting of MOVE and EOVE

The atmosphere in a glass container equipped with a three-way stopcock was replaced with nitrogen, and the glass container was heated at 250° C. under a nitrogen atmosphere to remove water adsorbed on the inside surface of the container. After cooling the system to room temperature, 12 mmol of MOVE, 16 mmol of ethyl acetate, 0.1 mmol of 1-isobutoxyethyl acetate and 11 ml of toluene were added to the system, and upon the system reached 0° C., 0.1 g (0.2 mmol) of 25 wt % ethyl aluminum sesquichloride/toluene solution to initiate polymerization. Thus the A component of the AB block polymer was synthesized.

The molecular weight of the A component was monitored with time by molecular sieve column chromatography (GPC), and after the completion of the A component polymerization, 12 mmol of EOVE as a B component was added to synthesize the AB diblock polymer. The polymerization reaction was stopped by the addition of 0.3 wt % ammonia/methanol solution to the system. The mixed solution after the reaction was diluted with dichloromethane, washed with 0.6 N hydrochloric acid solution three times and with distilled water three times, and concentrated and dried on an evaporator, and then vacuum dried to obtain the object MOVE-EOVE diblock polymer. The resultant compound was identified by NMR and GPC analyses, and satisfactory spectra were obtained in both analyses (Mn=2.5×10⁴, Mw/Mn=1.3).

### Preparation of Pigment Dispersion Ink

A pigment, a nonionic amphipathic block polymer, an ionic polymer and diethylene glycol were added to ion-exchanged water and the pigment was dispersed with an ultrasonic homogenizer. The dispersion was centrifuged (20,000 rpm×20 min) to remove coarse particles, so that a pigment dispersed solution was obtained.

Adequate amounts of aqueous solvent, ion-exchanged water and additives were added to the above pigment dispersed solution, and the mixtures were pressure-filtered through a 1 μm filter to prepare various types of stimulation-responsive ink jet ink (total 100 parts). The composition of the resultant ink (Sample α) is shown below. During this operation, the mixtures were appropriately heated or cooled.

Sample α

| | |
|---|---|
| Carbon black (Cabot, Mogul L) | 5 parts |
| MOVE-b-EOVE | 4 parts |
| (MOVE: EOVE = 1: 1, Mw/Mn = 1.3) | |
| Diethylene glycol | 4 parts |
| Polystyrene polysodium acrylate block polymer | 2 parts |
| (number average molecular weight: 10160, | |
| Mw/Mn = 1: 1, polymerization rate: about 1: 1) | |
| Ion-exchanged water | 85 parts |

An ink tank of an ink jet recording apparatus (Canon, BJC-800 J) was filled with the sample α and ink jet recording was performed on recycled paper. As a result, clear print of black characters was obtained. The pigment was neither aggregated nor precipitated and was dispersed well in the ink even after the ink was allowed to stand for 1 week. A 50-fold dilution of the ink was prepared with distilled water and the particle size was measured with a dynamic light scattering measuring device, DLS 7000 (Otsuka Electronics). The average particle size (diameter) obtained was 321 nm. After 3-month-storage at room temperature, no precipitate was observed in the ink.

### Example 2

Recording of black and yellow stripes was performed on recycled paper with the same ink jet recording apparatus (Canon, BJC-800 J) using the ink of Example 1 and an ink (1 wt % of Direct Yellow -12, 10 wt % of ethylene glycol, 2 wt % of aluminum nitrate and 87 wt % of distilled water). Satisfactory recording was done almost free from blur at the boundaries between the black and yellow stripes.

### Example 3

A pH 4 solution of 5 wt % polyacrylic acid in water was sprayed over a recording medium of plain paper, to prepare a recording medium having stimulating action. Then, ink jet recording was performed using the ink of Example 1 in the same manner as described above. As a result, fine print was obtained. After 3 minutes, the print was overwritten with a line marker, but no tail of black ink was observed.

### Comparative Example 1

An ink composition was prepared in the same manner as in Example 1, except that the nonionic amphipathic block polymer was omitted from the composition. A 50-fold dilution of the composition was prepared with distilled water and the particle size was measured with a dynamic light scattering measuring device, DLS 7000 (Otsuka Electronics). The average particle size (diameter) obtained was 512 nm. After 3-month-storage at room temperature, black precipitates were observed in the ink.

### Example 4

A diblock polymer (Mn=3.5×10⁴, Mw/Mn=1.3) with polymerization ratio of 100:90 was synthesized from isobutyl vinyl ether and 2-methoxyethyl vinyl ether in the same manner as in Example 1.

Separately, a triblock polymer with polymerization ratio of 100:90:30 was synthesized from isobutyl vinyl ether, 2-methoxyethyl vinyl ether and 4-(2-vinyloxy)ethoxy ethyl benzoate in the same manner as in Example 1. The triblock polymer was hydrolyzed to make the ester portion to sodium carboxylate.

Two parts by weight of the diblock polymer, 3 parts by weight of the sodium-carboxylate-type triblock polymer and 2 parts by weight of an oil-soluble dye Oil Blue N (Sigma-Aldrich), were made consolute in 30 parts by weight of DMF, and 200 parts by weight of distilled water was added to the solution to prepare a dispersion of polymer micelles in which Oil Blue N was included. Measurements with a dynamic light scattering measuring device showed that the particle size was 87 nm, and the index of dispersion degree (the index of dispersion degree μ/G² (μ: a quadratic coefficient of Cumulant Expansion, G: attenuation constant) presented by Gulari et al. is commonly used as an index of particle size uniformity (“The Journal of Chemical Physics” vol. 70, 3965, 1979)) was 0.05. Printing was performed with BJC-800 J, whose ink tank was filled with this composition, in the same manner as in Example 1, and fine print was obtained. After 5 minutes of printing, the print was rubbed hard with a commercially available line marker three times, but no tail of blue ink was observed.

### Example 5

A diblock polymer and a sodium-carboxylate-type triblock polymer were synthesized in the same manner as in Example 4, except that 2-ethoxyethyl vinyl ether was used instead of isobutyl vinyl ether. A dispersion of polymer micelles in which Oil Blue N (Aldrich) was included was prepared using these polymers in the same manner as in Example 4. Printing test was conducted with an ink jet recording apparatus (Canon, BJC-800 J), whose ink tank was filled with this composition, in the same manner as in Example 4, and fine print was obtained. After 5 minutes of printing, the print was rubbed hard with a commercially available line marker three times, but no tail of blue ink was observed.

It has been proved that when this composition is cooled to 0° C., the hydrophobic polymerized portion of 2-ethoxyethyl vinyl ether becomes hydrophilic and the micelles disintegrate. When actually cooling the composition to 0° C., the Oil Blue N was released and the solution was decolorized. This indicated that the Oil Blue N was included in polymer micelles.

What is claimed is:

1. A functional composition comprising a nonionic amphipathic block polymer, an ionic polymer, a functional substance and a solvent.

2. The functional composition according to claim 1, wherein said ionic polymer is a block polymer.

3. The functional composition according to claim 2, wherein at least one block segment of said amphipathic block polymer and that of said ionic block polymer have a same repeated structural unit.

4. The functional composition according to claim 2, wherein said amphipathic block polymer and said ionic block polymer form micelles.

5. The functional composition according to claim 2, wherein said functional substance is included in at least one of said amphipathic block polymer and said ionic block polymer.

6. The functional composition according to claim 2, wherein said ionic polymer is an amphipathic block polymer.

7. The functional composition according to claim 1, wherein said functional composition has a stimulation-responsiveness.

8. The functional composition according to claim 1, wherein said amphipathic block polymer and said ionic block polymer have a different stimulation-responsiveness.

9. The functional composition according to claim 1, wherein said amphipathic block polymer comprises a polyvinyl ether backbone structure.

10. The functional composition according to claim 9, wherein said polyvinyl ether backbone structure includes one of the following general formula (1): 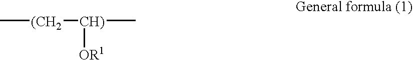
wherein R¹ is an alkyl group or one selected from the group consisting of —(CH(R²)—CH(R³)—O)ₗ—R⁴ and —(CH₂)ₘ—(O)ₙ—R⁴; and in the above groups, l is an integer selected from 1 to 18, m is an integer selected from 1 to 36, n is 0 or 1, R² and R³ are independently H or CH₃, R⁴ is selected from the group consisting of H, a straight-chain, branched-chain or cyclic alkyl group of 1 to 18 carbon atoms, an aromatic ring, —CO—CH═CH₂ and —CO—C(CH₃)═CH₂, and when R⁴ is any one other than a hydrogen atom, the carbon atoms are substituted or not substituted with a straight-chain or branched-chain alkyl group with 1 to 4 carbon atoms respectively, and a carbon atom in the aromatic ring is replaced or not replaced with nitrogen atoms respectively.

11. An ink composition comprising a nonionic amphipathic block polymer, an ionic polymer, a coloring material and a solvent.

12. A method of providing a liquid composition to a recording medium comprising the steps of:
preparing a liquid composition; and
providing the composition to a recording medium, wherein the composition comprises a nonionic amphipathic block polymer, an ionic polymer, a functional substance and a solvent.

13. The liquid composition providing method according to claim 12, further comprising a step of stimulating the composition, wherein the composition is fixed on the recording medium by the stimulation.

14. The liquid composition providing method according to claim 13, wherein the step of stimulating the composition is a step of bringing the composition into contact with a substance or a composition for stimulating the composition.

15. An apparatus for providing a liquid composition to a medium comprising
means for providing a liquid composition to a recording medium by adding energy to the composition; and
means for driving the composition-providing means,
wherein the composition comprises a nonionic amphipathic block polymer, an ionic polymer, a functional substance and a solvent.

16. The apparatus according to claim 15, further comprising means for stimulating the composition, wherein the composition is fixed on the recording medium by the stimulation.

17. The apparatus according to claim 16, wherein the means for stimulating the composition is means for bringing the composition into contact with a substance or composition for stimulating the composition.
